(19) United States
(12) Patent Application Publication
(10) Pub. No.: US 2005/0020593 A1
(43) Pub. Date: Jan. 27, 2005

(54) Aryl-heteroaromatic compounds, compositions comprising them and use

(75) Inventors: Patrick Mailliet, Fontenay Sous Bois (FR); Alain Le-Brun, Vigneux (FR); Fabienne Thompson, Paris (FR); Gilles Tiraboschi, Chevilly Larue (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma, Antony (FR)

(21) Appl. No.: 10/898,517

(22) Filed: Jul. 23, 2004

Related U.S. Application Data
(60) Provisional application No. 60/505,184, filed on Sep. 23, 2003.

(30) Foreign Application Priority Data
Jul. 24, 2003 (FR) 0309092

(51) Int. Cl. A61K 31/53; A61K 31/52; A61K 31/496

(52) U.S. Cl. 514/243; 514/248; 514/249; 514/252.16; 514/253.04; 514/254.09; 544/183; 544/235; 514/277; 514/362; 544/373

(57) ABSTRACT

A series of aryl-heteroaromatic compounds, compositions comprising them and use thereof are disclosed and claimed. The present invention relates in particular to novel aryl-heteroaromatic compounds exhibiting anticancer activity, and in particular inhibitory activity with regard to tubulin polymerization.

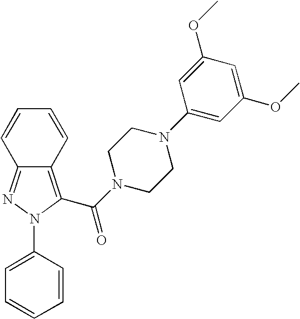

This application claims the benefit of U.S. Provisional Application No. 60/505,184, filed Sep. 23, 2003 and benefit of priority of French Patent Application No. 03/09,092, filed Jul. 24, 2003, both of which are incorporated herein by reference in their entirety.

### BACKGROUND OF THE INVENTION

1. Field of the Invention

The present invention relates to novel chemical compounds, particularly novel aryl-heteroaromatic compounds, to compositions comprising them and to their use as medicinal products.

2. Description of the Art

More particularly, according to a first aspect, the invention relates to novel aryl-heteroaromatic products exhibiting anticancer activity, and in particular inhibitory activity with regard to tubulin polymerization.

The bicyclic aryl-heteroaromatic products concerned with here correspond to formula (I) below:

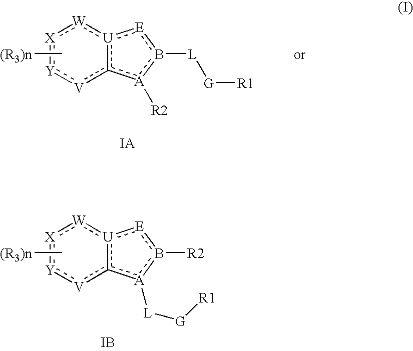

Some bicyclic aryl-heteroaromatic products corresponding to formula (I) are known:

BE 849627 (Hoechst) claims 2-amino-3-carbonylindole derivatives having an activity on cardiac circulation. An example of a product in which R2=4-(aryl/heteroaryl)piperidinyl and R1=aryl/heteroaryl is neither presented nor suggested.

WO 03/037862 (Nippon Shinyaku) claims the preparation of derivatives of indole amides and of pyrrolo[2,3-b]pyridine that are useful as TGF-β (transforming growth factor-β) antagonists. These products are useful for treating osteoporosis. The products disclosed by WO 03/037862 are not part of the invention.

WO 01/43746 (Nippon Shinyaku) (equivalent to EP 1243268) claims the use of indoles substituted with amides for treating nephritis. Use of these products in oncology is neither claimed nor mentioned. All the products described in WO 01/43746 were already disclosed in WO 00/44743, below.

WO 00/44743 (Nippon Shinyaku) (equivalent to EP 1156045) claims antagonists and inhibitors of production of TGF-β, that are useful for treating osteoporosis or pruritis, comprising known and novel indol-3-ylcarboxamide derivatives. Use of these products in oncology is neither claimed nor mentioned.

EP 624584 (Daichi) claims piperazine derivatives that are useful as calmodulin inhibitors, for treating diseases such as ischaemia, hypoxia or certain diseases related to the central nervous system.

EP 1314733 (Aventis) claims in particular indoles substituted in the 2-position with an N-carbonylpiperazine, for use in the cardiovascular field. Use in oncology is claimed, although no demonstration of the anticancer activity is presented. In the examples of EP1314733, when G is piperidine, R1 is never aryl, but alkyl substituted with aryl or heteroaryl. However, the products according to the invention, described below, cannot have a substituent R1 which is alkyl substituted with aryl or heteroaryl, which is optionally substituted, without suffering loss of biological activity. The products according to the invention have a substituent R1 which is exclusively aryl or heteroaryl, which is optionally substituted. This comment applies mutatis mutandis to the substituent R2.

All of the references described herein are incorporated herein by reference in their entirety.

### SUMMARY OF THE INVENTION

Now, surprisingly, it has been found that products corresponding to formula (I) below exhibit considerable inhibitory activity with regard to tubulin polymerization: 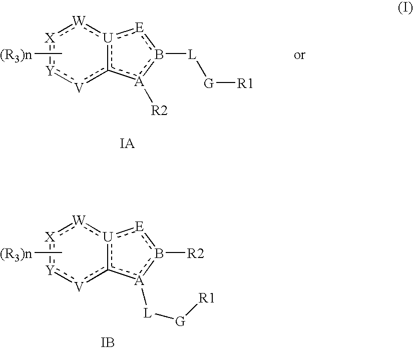
in which:
1) (i) A, B, U, V, W, X, Y may be N, C or CR4; or
(ii) A, B, U may be N, C or CR4; V and W are CH₂, X is chosen from S, SO and SO₂; and Y is a bond;
2) L-G-R1 is chosen from 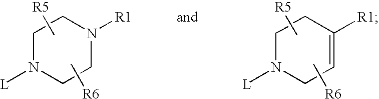
3) E is CR4, N, NR4 or S;
4) R1 and R2 are selected independently from the group consisting of aryl, heteroaryl, substituted aryl and substituted heteroaryl;
5) L is selected from the group consisting of C═O, C═S and C═N(R7);
6) R3 is selected from the group consisting of halogen, CF₃, CN, NO₂, (C₁-C₃)alkyl, (C₁-C₃)alkenyl, (C₁-C₃)alkynyl, O—R7, S—R7, SO—R7, SO₂—(R7), N(R7)(R8), halogen, CO—OR7, CO—N(R7)(R8), SO₂—N(R7)(R8), NR7-CO—R8 and NR7-SO₂—(C₁-C₃)alkyl;
7) n=0, 1, 2 or 3, it being understood that, when n is greater than 1, the radicals R3 may be identical or different, and when n=2, X and Y are not simultaneously substituted with R3;
8) R4 is selected from the group consisting of H and (C₁-C₃)alkyl;
9) R5 and R6 are selected independently from the group consisting of H and (C₁-C₃)alkyl;
10) R7 and R8 are selected independently from the group consisting of H, (C₁-C₃)alkyl and substituted (C₁-C₃)alkyl;
   in the racemic form, enriched in one enantiomer, enriched in one diastereoisomer, its tautomers, its prodrugs and its pharmaceutically acceptable salts, with the proviso that the product of formula (I) is not one of the following compounds (optionally salified): 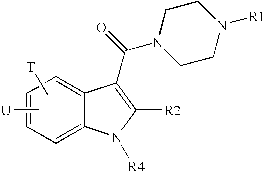
   in which
(i) R1 is chosen from pyrid-2-yl and substituted pyrid-2-yl, each optionally in N-oxide form;
R2 is chosen from thien-2-yl, pyrid-2-yl, pyrid-3-yl, pyrid-4-yl, phenyl, phenyl substituted with at least one substituent chosen from F, OH, CF₃, Me, OMe and NO₂, in which, when R2 is pyrid-2-yl, pyrid-3-yl or pyrid-4-yl, R2 may be present in N-oxide form;
R4 is chosen from methyl, 2-fluoroethyl and ethyl;
T and U are chosen independently from H, methyl, Cl and F; or else
(ii) R1 is chosen from pyrid-3-yl and pyrid-4-yl,
R2 is chosen from thien-2-yl and phenyl;
R4 is chosen from methyl and 2-fluoroethyl;
T and U are chosen independently from H, methyl, Cl and F;
(iii) R1 is pyrid-2-yl substituted in the 5-position with a tetrazolyl or amide substituent, which is optionally substituted;
R2 is phenyl;
R4 is methyl; T is 5-methyl; U is H;
(iv) R1 is pyrazin-2-yl substituted in the 5-position with CH₂CONH₂ or amide, which is optionally substituted;
R2 is phenyl;
R4 is methyl; T is chosen from 5-methyl, 5-chloro, 5-fluoro and 5-bromo; U is H; 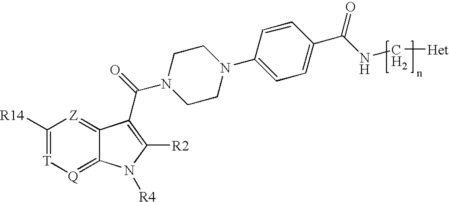
   in which:
n is 2 or 3;
Het is 4-methylthiazol-5-yl or imidazol-1-yl;
R2 is phenyl;
R4 is methyl;
T, Q and Z are chosen independently from N and CH, and R14 is H or methyl; in which:
   when T is N, then Q and Z are CH and R14 is H;
   when Q is N, and T and Z are CH, then R14 is H or methyl;
and
   when T is CH, then R14 is H.

### DETAILED DESCRIPTION OF THE INVENTION

Products of formula (I) 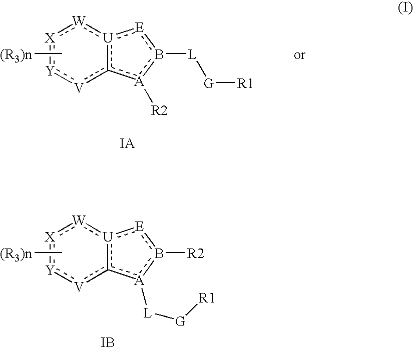
in which L-G-R1 is chosen from 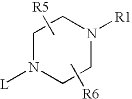
are preferred.

Products of formula (IA) for which:
A is N, B is C and E is CR4, with R4 being H, are preferred.

Products of formula (IB), for which:
A is C, B is N and E is NR4, with R4 being H, are preferred.

Products of formula (I) for which:
U=N; A and B=C; E=CH; V and W are CH₂, X is SO₂; and Y is a bond are preferred.

A preferred substituent R1 may be chosen from phenyl, phenyl substituted with at least one radical chosen from halogen, CF₃, CN, NO₂, (C₁-C₃)alkyl, O—R10, S—R10, N(R10)(R11), CO—O—R10, CO—N(R10)(R11) and NH—CO—R10 in which R10 and R11 are chosen independently from H, (C₁-C₃)alkyl, halogenated (C₁-C₃)alkyl, (C₁-C₃)alkyl-OH, (C₁-C₃)alkyl-NH₂, (C₁-C₃)alkyl-COOH, (C₁-C₃)alkyl-OCH₃, (C₁-C₃)alkyl-NHCH₃, pyridyl and pyridyl substituted with at least one radical chosen from halogen, (C₁-C₃)alkyl, O—R12, S—R12 and N(R12)(R13), in which R12 and R13 are chosen independently from H and (C₁-C₃)alkyl.

More preferably, R1 is phenyl substituted in the 3-position with halogen or (C₁-C₃)alkyl, (C₁-C₃)alkoxy, (C₁-C₃)amino, CONH₂, CO—NH—CH₂)₂—OH or NH—CO—CH₃; or 3-pyridyl; 2- or 3-pyridyl substituted with halogen, (C₁-C₃)alkyl or (C₁-C₃)alkoxy.

When R1 is substituted phenyl, preferred substitution combinations may be chosen from 2,3-disubstituted phenyl, 2,5-disubstituted phenyl, 3-substituted phenyl, 3,5-disubstituted phenyl and 3,4-disubstituted phenyl, more preferably from 3-substituted phenyl, 3,5-disubstituted phenyl and 3,4-disubstituted phenyl.

When R1 is 2-pyridyl, preferred substitutions are chosen from 4- or 6-substituted 2-pyridyl and 4,6-disubstituted 2-pyridyl.

When R1 is 3-pyridyl, preferred substitutions are 2- or 5-substituted 3-pyridyl.

Very preferably, R1 is phenyl substituted in the 3-position with a chloro radical or a cyano radical or a carboxamido radical or a methanol radical, or in the 3- and 5-positions with two methoxy radicals.

A preferred substituent R2 may be chosen from phenyl, phenyl substituted with at least one radical chosen from halogen, alkyl, O—R10, S—R10, N(R10)(R11), in which R10 and R11 are chosen independently from H, alkyl and halogenated alkyl; or 3-pyridyl.

According to a second aspect, the invention relates to pharmaceutical compositions comprising a product according to its first aspect, in combination with a pharmaceutically acceptable excipient.

A product according to the invention can advantageously be used as an agent which inhibits tubulin polymerization, as an agent which inhibits the proliferation of tumor cells, for promoting the breakup of clusters of cells originating from a vascular tissue, or for producing a medicinal product of use in treating a pathological condition, preferably cancer.

In general, the invention relates to the use of a product of formula (I) below: 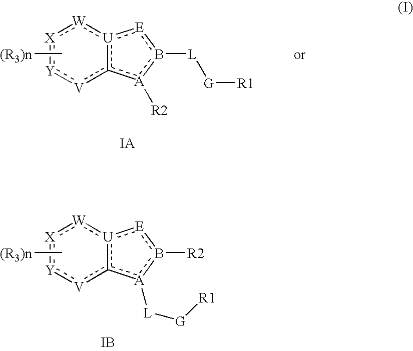
in which:
1) (i) A, B, U, V, W, X, Y may be N, C, or CR4; or
(ii) A, B, U may be N, C or CR4; V and W are CH2, X is chosen from S, SO and SO2; and Y is a bond;
2) L-G-R1 is chosen from 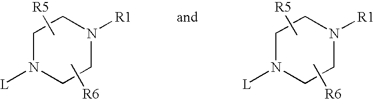
3) E is CR4, N, NR4 or S;
4) R1 and R2 are selected independently from the group consisting of aryl, heteroaryl, substituted aryl and substituted heteroaryl;
5) L is selected from the group consisting of C═O, C═S and C═N(R7);
6) R3 is selected from the group consisting of halogen, CF₃, CN, NO₂, (C₁-C₃)alkyl, (C₁-C₃)alkenyl, (C₁-C₃)alkynyl, O—R7, S—R7, SO—R7, SO₂—(R7), N(R7)(R8), halogen, CO—OR7, CO—N(R7)(R8), SO₂—N(R7)(R8), NR7-CO—R8 and NR7-SO₂—(C₁-C₃)alkyl;
7) n=0, 1, 2 or 3, it being understood that, when n is greater than 1, the radicals R3 may be identical or different, and when n=2, X and Y are not simultaneously substituted with R3;
8) R4 is selected from the group consisting of H and (C₁-C₃)alkyl;
9) R5 and R6 are selected independently from the group consisting of H and (C₁-C₃)alkyl;
10) R7 and R8 are selected independently from the group consisting of H, (C₁-C₃)alkyl and substituted (C₁-C₃)alkyl;
   in the racemic form, enriched in one enantiomer, enriched in one diastereoisomer, its tautomers, its prodrugs and its pharmaceutically acceptable salts,
(i) as an agent which inhibits tubulin polymerization,
(ii) as an agent which inhibits the proliferation of tumor cells,
(iii) for promoting the breakup of clusters of cells originating from a vascular tissue, and/or
(iv) for treating cancer.

In general, products of formula (IAa), (Iab), (Iba) or (IBb) in accordance with the invention, in which L is C(O), can be prepared by coupling a heteroarylcarboxylic acid substituted in the position ortho to the carboxyl function with an aryl or heteroaryl radical, of formula (IIA) or (IIB), in which A, B, U, V, W, X, Y, E and R2 are defined as above, with respectively a piperazine derivative of formula (Illa) or a 1,2,3,6-tetrahydropyridine derivative (IIIb), in which R1 is defined as above, according to scheme 1:

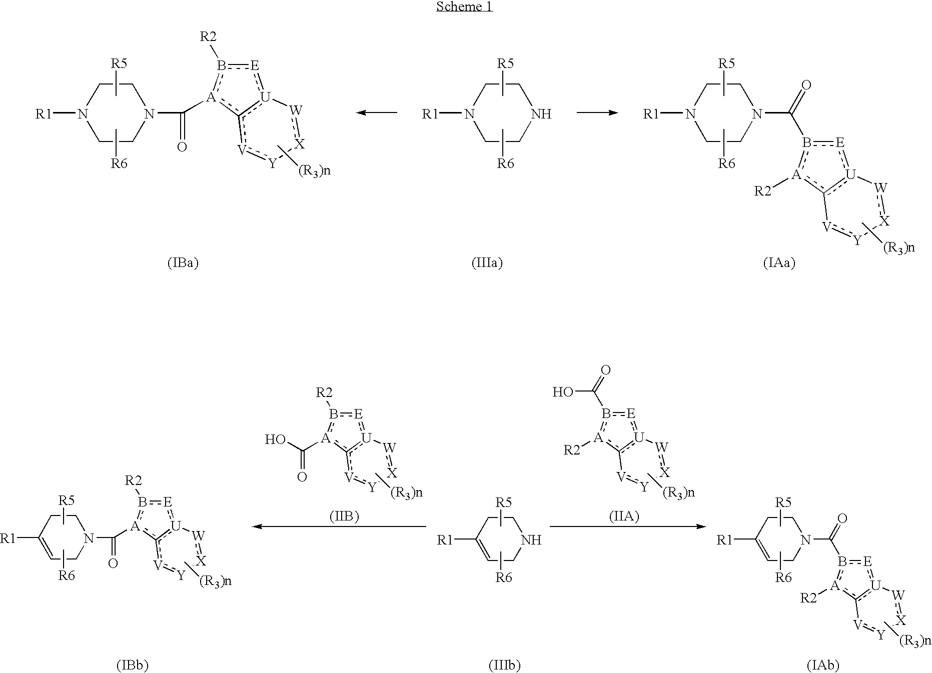

The heteroarylcarboxylic acids of formula (IIA) or (IIB) in which A, B, U, V, W, X, Y, E and R2 are commercially available or can be obtained according to general synthetic methods known to those skilled in the art.

The piperazine derivatives of formula (IIIa), in which R1, R5 and R6 are defined as above, are either commercially available or are prepared according to conventional methods known to those skilled in the art.

Among these methods, N1-aryl(heteroaryl)ation, according to scheme 2, of piperazines carrying a protective group on 4-nitrogen is particularly advantageous in the context of the invention:

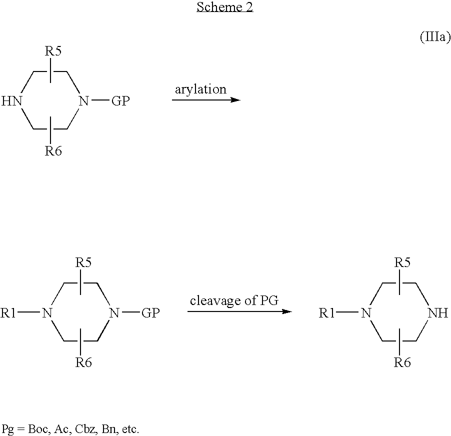

The reaction of aryl(heteroaryl)ation of piperazines, generally of Hartwig/Büchwald type, can be carried out according to the conditions described in Biorg. Med. Chem. Lett., 11, 1375 (2001) or in Biorg. Med. Chem., 10, 3817 (2002).

Another method for the synthesis of aryl(heteroaryl)piperazines, that is particularly advantageous in the context of the invention, when R5 and R6 represent hydrogen atoms, consists of the reaction of an aryl(heteroaryl)amine with a bis(2-hydroxy- or 2-haloethyl)amine, at a temperature of greater than 100-120° C. according to scheme 3:

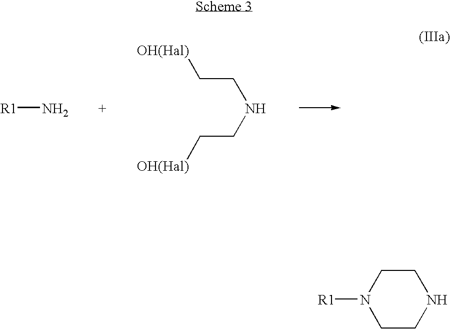

It is particularly advantageous to carry out the reaction in the presence of microwaves under the conditions described in Synth. Comm., 28, 1175 (1998) or in Tetrahedron Lett, 38, 6875 (1997).

The 1,2,3,6-tetrahydropyridine derivatives (IIIb) in which R1, R5 and R6 are defined as above are either commercially available or are prepared according to conventional methods known to those skilled in the art.

Among these methods, the action, according to scheme 4, of an organometallic aryl(heteroaryl) derivative, such as an organomagnesium derivative, an organolithium derivative or an organocerium derivative, on a piperidin-4-one derivative, the nitrogen atom of which is substituted with a protective group, is particularly advantageous.

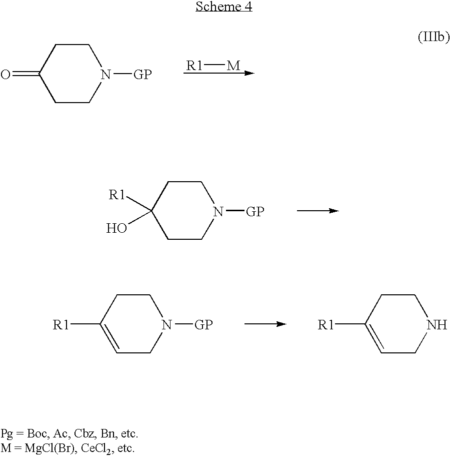

It is possible in particular to carry out the reaction under the conditions described in J. Med. Chem., 38, 1998 (1995) or in EP 306764 or in J. Med. Chem., 28, 311 (1985).

When R5 and R6 represent hydrogen atoms, the coupling of Suzuki type of the pinacol ester of N-Boc-1,2,3,6-tetrahydropyridyl-4-boronic acid with an aryl or heteroaryl halide, preferably a bromide or an iodide, under the conditions described in Tetrahedron Lett, 41, 3705 (2000), according to scheme 5, is particularly advantageous in the context of the invention: it is understood that the Boc protective group can be replaced with any other protective group compatible with the reaction conditions and that the pinacolboronic ester can also be replaced with any other boron derivative, acid or ester, compatible with said conditions.

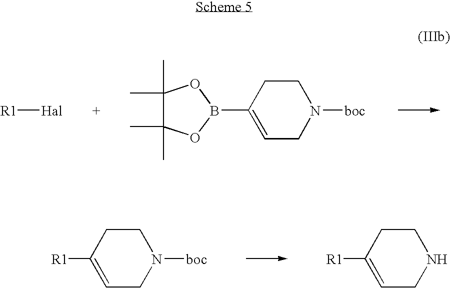

In general, products of formula (IAa), (IAb), (IBa) or (IBb) in accordance with the invention in which L is C(S) can be prepared by thionation of a compound of formula (IAa), (IAb), (IBa) or (IBb), respectively, in which L is C(O), by any one of the reduction methods known to those skilled in the art. It is particularly advantageous, in the context of the invention, to carry out the thionation using Lawesson's reagent, the reaction being carried out according to Bull. Soc. Chim. Belg., 87, 293 (1978).

In general, products of formula (Ia) or (Ib) in accordance with the invention in which L is C(NH) can be prepared from the nitriles derived from the products of formula (II), using various methods known to those skilled in the art. It is generally necessary to activate the not very reactive nitrile, either with aluminum chloride, the reaction being carried out according to J. Chem. Soc. 1947, 1110; or with cuprous iodide, the reaction being carried out according to Tetrahedron Lett., 34, 6395 (1993); or by converting nitrile to iminoether prior to the reaction with the piperazine or 1,2,3,6-tetrahydropyridine or piperidine derivative, the reaction being carried out according to Eur. J. Med. Chem., 24, 427 (1989).

In general, products of formula (Ia) in accordance with the invention in which L is C(NR7), with R7 the same as or different from the hydrogen atom, can be prepared from the products of formula (Ia), in which L is C(O) and/or C(S), using the various methods known to those skilled in the art. In the context of the invention, when L is C(O), it is particularly advantageous to successively react oxalyl chloride, and then an amine R7-NH₂, the reaction being carried out according to Pol. J. Chem., 58, 117 (1984), and, when L is C(S), it is particularly advantageous to react first methyl iodide and then an amine R7-NH₂, the reaction being carried out according to Eur. J. Med. Chem, 12, 365 (1977).

More specifically and more particularly advantageously in the context of the invention, products in accordance with the invention can also be prepared on a solid phase, according to reaction scheme 6:

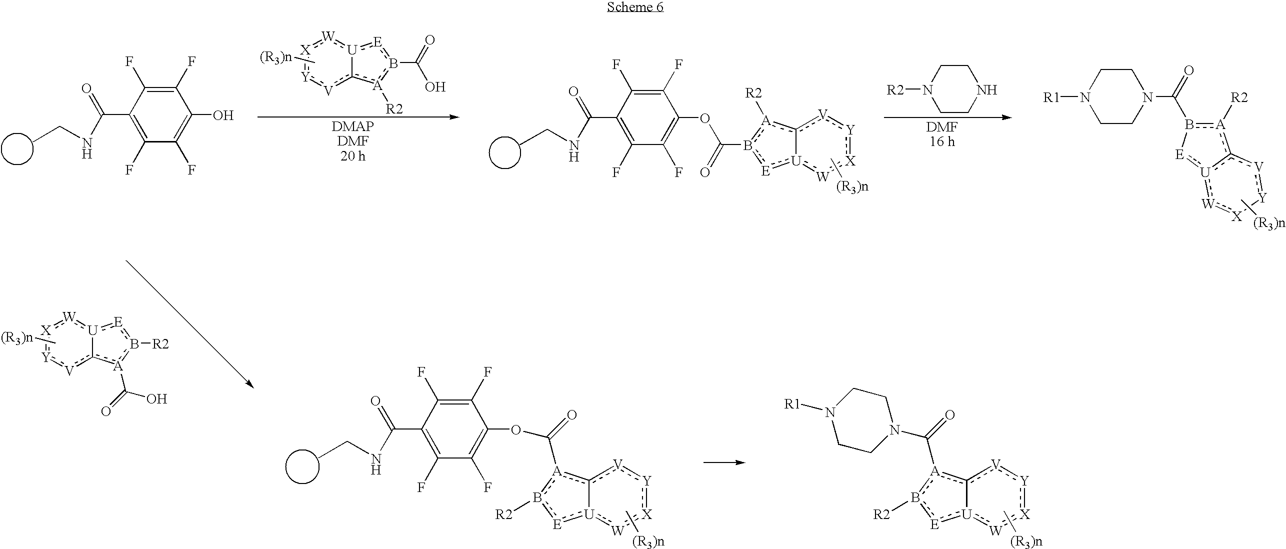

The general synthetic methods presented, in particular those described in schemes 1 to 6, illustrate, without implied limitation, possible preparations of the compounds of the invention. Many other synthetic pathways can be used, in particular those described in: Comprehensive Heterocyclic Chemistry, by A. Katritsky et al. (Pergamon Press).

The examples below illustrate, without implied limitation, the products of the invention. The various products are purified either as described in the examples or by LC/MS under the general conditions described below:

Purification by LC/MS:

The products were purified by LC/MS using a Waters FractionsLynx system composed of a Waters model 600 gradient pump, a Waters model 515 regeneration pump, a Waters Reagent Manager dilution pump, a Waters model 2700 auto-injector, two Rheodyne model LabPro valves, a Waters model 996 diode array detector, a Waters model ZMD mass spectrometer and a Gilson model 204 fraction collector. The system was controlled by the Waters FractionLynx software. Separation was carried out alternately on two Waters Symmetry columns (C₁₈, 5 μM, 19×50 mm, catalogue reference 186000210), one column undergoing regeneration with a 95/5 (v/v) water/acetonitrile mixture comprising 0.07% (v/v) of trifluoroacetic acid, while the other column was being used for separation. The columns were eluted using a linear gradient of from 5 to 95% of acetonitrile comprising 0.7% (v/v) of trifluoroacetic acid in water comprising 0.07% (v/v) of trifluoroacetic acid), at a flow rate of 10 ml/min. At the outlet of the separation column, one thousandth of the effluent is separated by means of an LC Packing Accurate, diluted with methyl alcohol at a flow rate of 0.5 ml/min and sent to the detectors, in a proportion of 75% to the diode array detector and the remaining 25% to the mass spectrometer. The rest of the effluent (999/1000) is sent to the fraction collector, where the flow is discarded for as long as the mass of expected product is not detected by the FractionLynx software. The molecular formulae of the expected products are supplied to the FractionLynx software, which actuates the collection of the product when the mass signal detected corresponds to the ion [M+H]⁺ and/or to the [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, the collection is also actuated when the mass signal of the ion [M+2H]⁺⁺ and/or [M+Na+H]⁺⁺ are detected. The products were collected in a tared glass tube. After collection, the solvents were evaporated in a Savant AES 2000 or Genevac HT 8 centrifugal evaporator and the masses of products were determined by weighing the tubes after evaporation of the solvents.

The LC/MS analyses were carried out on a Micromass model LCT device connected to an HP 1100 device. The abundance of the products was measured using an HP G1315A diode array detector over a wavelength range of 200-600 nm and a Sedex 65 light scattering detector. The mass spectra were acquired over a range of 180 to 800. The data were analyzed using the Micromass MassLynx software. Separation was carried out on a Hypersil BDS C18, 3 μm (50×4.6 mm) column, by eluting with a linear gradient of from 5 to 90% of acetonitrile comprising 0.05% (v/v) of trifluoroacetic acid (TFA) in water comprising 0.05% (v/v) TFA, over 3.5 min at a flow rate of 1 ml/min. The total analysis time, including the period for re-equilibrating the column, is 7 min.

### EXAMPLE 1

### [4-(3-chlorophenyl)piperazin-1-yl](1-phenyl-1H-indol-2-yl)methanone

217 μl of oxalyl chloride and a few drops of dimethylformamide are successively added to a solution of 0.5 g of 1-phenylindol-2-carboxylic acid, which can be prepared according to Pharmazie (2002) 57, 238-42, 10 ml of dichloromethane in a 25 ml three-necked flask under an argon atmosphere, and stirring is carried out at ambient temperature for 2 hours. The solution thus obtained is transferred into a dropping funnel and is added dropwise to a solution, cooled to 0° C. under an argon atmosphere, of 431 mg of 1-(3-chlorophenyl)piperazine in 5 ml of dichloromethane comprising 355 μl of triethylamine. After stirring at ambient temperature for 20 hours, 20 ml of water is added, and the organic phase is separated by settling out, washed with water, dried over magnesium sulfate, and concentrated under reduced pressure. The residue is purified by recrystallization from a mixture of methanol and ethanol (20-80 by volume). 400 mg of [4-(3-chlorophenyl)-piperazin-1-yl](1-phenylindol-2-yl)methanone are thus obtained in the form of white crystals, the characteristics of which are as follows
melting point (Kofler bench)=168° C.)
¹H NMR spectrum (400 MHz, d6-(CD₃)₂SO, at a temperature of 353K, δ in ppm): 3.08 (mt: 4H), 3.61 (t, J=5 Hz: 4H); 6.82 (dd, J=8 and 1.5 Hz: 1 H); 6.86 (dd, J=8 and 2 Hz: 1H); 6.91 (mt: 2H); from 7.20 to 7.35 (mt: 3H); 7.35 (broad d, J=8 Hz: 1H); from 7.40 to 7.50 (mt: 3H); 7.59 (broad t, J=7.5 Hz: 2H); 7.74 (d, J=8 Hz: 1H).

### EXAMPLE 2

### [4-(3-chlorophenyl)piperazin-1-yl](1-phenylindazol-3-yl)-methanone

Stage 1: 114 mg of 2-phenyl-2H-indazole-3-carboxylic acid methyl ester, which can be prepared according to Acta Chem. Scand. (1999), 53, 814-23, are dissolved in 5 ml of ethanol in a 25 ml round-bottomed flask, and 0.94 ml of a 1M sodium hydroxide solution is added and then stirring is carried out at 60° C. for 21 hours. After concentration under reduced pressure, the reaction medium is taken up in 3.5 ml of water, 1.5 ml of an aqueous 1M hydrochloric acid solution are added and the mixture is left to crystallize for 3 hours. The crystals are filtered dried, washed 3 times with 1 ml of water and dried under vacuum at 50° C. 100 mg of 2-phenyl-2H-indazole-3-carboxylic acid are thus obtained in the form of a white solid which is used as it is in the subsequent stage.

Stage 2: 44.3 mg of 1-(3-dimethylaminopropyl)-3-ethylcarbodiimide hydrochloride (EDCl) and 2.8 mg of 1-hydroxybenzotriazole hydrate (HOBT) are added to a solution of 50 mg of 2-phenyl-2H-indazole-3-carboxylic acid in 5 ml of dichloromethane in a 25 ml three-necked flask under an argon atmosphere. After stirring for 10 minutes at ambient temperature, 45.4 mg of 1-(3-chlorophenyl)piperazine are added, and this reaction mixture is then stirred at ambient temperature for 24 hours. The reaction medium is diluted with 15 ml of dichloromethane and 5 ml of water. The organic phase is washed with water, dried over magnesium sulfate and concentrated under reduced pressure. The oily residue obtained is recrystallized from 5 ml of diethyl ether. 50.5 mg of [4-(3-chlorophenyl)piperazin-1-yl](2-phenyl-2H-indazol-3-yl)methanone are thus obtained in the form of white crystals, the characteristics of which are as follows:
melting point (Kofler bench): 181°

### EXAMPLE 3

### [4-(3,5-dimethylphenyl)piperazin-1-yl](2-phenyl-2H-indazol-3-yl)methanone

The procedure is carried out as in stage 2 of Example 2, but using 50 mg of 2-phenyl-2H-indazole-3-carboxylic acid in 5 ml of dichloromethane, 44.3 mg of 1-(3-dimethylaminopropyl)-3-ethylcarbodiimide hydrochloride (EDCl), 2.8 mg of 1-hydroxybenzotriazole hydrate (HOBT) and 51.3 mg of 1-(3,5-dimethoxyphenyl)piperazine, at ambient temperature for 24 hours. After purification by flash chromatography on silica gel (70-230 mesh), elution being carried out with a mixture of dichloromethane and ethanol (98-2 by volume), 85 mg of [4-(3,5-dimethoxyphenyl)piperazin-1-yl](2-phenyl-2H-indazol-3-yl)methanone are obtained in the form of a white foam, the characteristics of which are as follows:
mass spectrum (EI): m/z=442 (M⁺)
¹H NMR spectrum (400 MHz, d6-(CD₃)₂SO, at a temperature of 373K, δ in ppm): 3.04 (unresolved peak: 4H); 3.57 (unresolved peak: 4H); 3.74 (s: 6H); from 6.00 to 6.10 (unresolved peak: 3H); 7.04 (broad t, J=7.5 Hz: 1H); 7.39 (broad dd, J=8 and 7.5 Hz: 1H); 7.45 (broad t, J=7.5 Hz: 1H); 7.51 (broad t, J=7.5 Hz: 2H); 7.66 (broad d, J=8 Hz: 1H); 7.82 (broad d, J=7.5 Hz: 2H); 8.74 (d, J=7.5 Hz: 1H).

### EXAMPLE 4

### [4-(3-chlorophenyl)piperazin-1-yl](2-phenylbenzo[b]thiophen-3-yl)methanone

The procedure is carried out as in stage 2 of Example 2, but using 100 mg of 2-phenylbenzo[b]thiophene-3-carboxylic acid, which can be prepared according to Monatsch Chem. (1969), 100, 899-904, in 20 ml of dichloromethane, 82.9 mg of 1-(3-dimethylaminopropyl)-3-ethylcarbodiimide hydrochloride (EDCl), 10.6 mg of 1-hydroxybenzotriazole hydrate (HOBT) and 77.3 mg of 1-(3-chlorophenyl)piperazine, at ambient temperature for 24 hours. After purification by flash chromatography on silica gel (70-230 mesh), elution being carried out with a mixture of cyclohexane and ethyl acetate (80-20 by volume), 110 mg of [4-(3-chlorophenyl)piperazin-1-yl](2-phenylbenzo[b]thiophen-3-yl)methanone are obtained in the form of a white foam, the characteristics of which are as follows:
mass spectrum (EI): m/z=432 (M⁺)
¹H NMR spectrum (300 MHz, d6-(CD₃)₂SO, δ in ppm): at ambient temperature, a mixture of rotamers is observed: 2.43 (mt: 1H); 2.97 (mt: 1H); from 3.00 to 3.20 (mt: 2H); from 3.15 to 3.45 (mt: 2H); 3.79 (mt: 1H); 3.90 (mt: 1H); from 6.75 to 6.85 (mt: 2H); 6.87 (t, J=2Hz: 1H); 7.20 (t, J=8 Hz: 1H); from 7.40 to 7.55 (mt: 3H); 7.53 (broad t, J=7.5 Hz: 2H); 7.63 (broad d, J=7.5 Hz: 2H); 7.70 (mt: 1H); 8.09 (mt: 1H).

### EXAMPLE 5

### [4-(3,5-dimethoxyphenyl)piperazin-1-yl](8-phenylindolizin-1-yl)-methanone

Stage 1: 359 mg of 8-phenylindolizine-1-carboxylic acid ethyl ester are dissolved in 15 ml of ethanol in a 25 ml round-bottomed flask, and 6.7 ml of a 1M sodium hydroxide solution are added and stirring is then carried out at reflux for 21 hours. After concentration under reduced pressure, the reaction medium is taken up in 40 ml of water, and 1.7 ml of an aqueous 5M hydrochloric acid solution are added. The precipitate formed is extracted with 3 times 25 ml of ethyl acetate, and drying over magnesium sulfate and concentration under vacuum are then performed. 143 mg of 8-phenylindolizine-1-carboxylic acid are thus obtained in the form of a khaki-beige foam, used as it is in the subsequent step.

Stage 2: 102.2 mg of 1-(3-dimethylaminopropyl)-3-ethylcarbodiimide hydrochloride (EDCl) and 13.1 mg of 1-hydroxybenzotriazole hydrate (HOBT) are added to a solution of 115 mg of 8-phenylindolizine-1-carboxylic acid in 10 ml of dichloromethane in a 25 ml three-necked flask under an argon atmosphere. After stirring at ambient temperature for 10 minutes, 107.7 mg of 1-(3,5-dimethoxyphenyl)piperazine are added, and this reaction mixture is then stirred at ambient temperature for 24 hours. The reaction medium is diluted with 15 ml of dichloromethane and 5 ml of water. The organic phase is washed with water, dried over magnesium sulfate and concentrated under reduced pressure. The residue is purified by flash chromatography on silica gel (70-230 mesh), elution being carried out with a mixture of cyclohexane and ethyl acetate (80-20 by volume). 117 mg of [4-(3,5-dimethoxyphenyl)piperazin-1-yl](8-phenylindolizin-1-yl)methanone are thus obtained in the form of a beige solid, the characteristics of which are as follows:
mass spectrum (EI): m/z=441 (M⁺)
¹H NMR spectrum (400 MHz, d6-(CD₃)₂SO, at a temperature of 373K, δ in ppm): 3.04 (unresolved peak: 4H); 3.57 (unresolved peak: 4H); 3.74 (s: 6H); from 6.00 to 6.10 (mt: 3H); 7.04 (broad t, J=7.5 Hz: 1H); 7.39 (broad dd, J=8 and 7.5 Hz: 1H); 7.45 (broad t, J=7.5 Hz: 1H); 7.51 (broad t, J=7.5 Hz: 2H); 7.66 (broad d, J=8 Hz: 1H); 7.82 (broad d, J=7.5 Hz: 2H); 8.74 (d, J=7.5 Hz: 1H).

### EXAMPLE 6

### [4-(3-chlorophenyl)piperazin-1-yl](8-phenylindolizin-1-yl)-methanone

By carrying out the procedure as in stage 2 of Example 5, but using 115 mg of 8-phenylindolizine-1-carboxylic acid, 102.2 mg of 1-(3-dimethylaminopropyl)-3-ethylcarbodiimide hydrochloride (EDCl), 13.1 mg of 1-hydroxybenzotriazole hydrate (HOBT) and 95.3 mg of 1-(3-chlorophenyl)-piperazine in 15 ml of dichloromethane; after purification by flash chromatography on silica gel (70-230 mesh), elution being carried out with a mixture of cyclohexane and ethyl acetate (80-20 by volume), 117 mg of [4-(3-chlorophenyl)piperazin-1-yl](8-phenylindolizin-1-yl)methanone are obtained in the form of a pale yellow solid, the characteristic of which is as follows:
mass spectrum (EI): m/z=415 (M⁺)

### EXAMPLE 7

### [4-(3-carboxamidophenyl)piperazin-1-yl](1-phenyl-1H-indol-2-yl)methanone

By carrying out the procedure as in stage 2 of Example 5, but using 237 mg of 1-phenyl-1H-indole-2-carboxylic acid, 211 mg of 1-(3-dimethylaminopropyl)-3-ethylcarbodiimide hydrochloride (EDCl), 13 mg of 1-hydroxybenzotriazole hydrate (HOBT) and 306 mg of 1-(3-carboxamido-phenyl)piperazine dihydrochloride in 15 ml of dichloromethane; after purification by flash chromatography on silica gel (70-230 mesh; elution being carried out with a mixture of dichloromethane and ethanol (97.5-2.5 by volume), 250 mg of [4-(3-carboxamidophenyl)piperazin-1-yl](1-phenyl-1H-indol-2-yl)methanone are obtained in the form of a white solid, the characteristic of which is as follows:
mass spectrum (EI): m/z=424 (M⁺)

### EXAMPLE 8

### [4-(3,5-dimethoxyphenyl)piperazin-1-yl](1-phenyl-1H-indol-2-yl)methanone

By carrying out the procedure as in stage 2 of Example 5, but using 237 mg of 1-phenyl-1H-indole-2-carboxylic acid, 211 mg of 1-(3-dimethylaminopropyl)-3-ethylcarbodiimide hydrochloride (EDCl), 13 mg of 1-hydroxybenzotriazole hydrate (HOBT) and 244 mg of 1-(3,5-dimethoxyphenyl)piperazine in 15 ml of dichloromethane; after purification by flash chromatography on silica gel (70-230 mesh), elution being carried out with a mixture of dichloromethane and ethanol (98.5-1.5 by volume), followed by recrystallization from 10 ml of diethyl ether, 350 mg of [4-(3,5-dimethoxyphenyl)piperazin-1-yl](1-phenyl-1H-indol-2-yl)methanone are obtained in the form of white crystals, the characteristics of which are as follows:
mass spectrum (EI): m/z=441 (M⁺)
melting point (Kofler bench)=146° C.

### EXAMPLE 9

### [4-(3,5-dimethoxyphenyl)piperazin-1-yl](5-methoxy-1-phenyl-1H-indol-2-yl)methanone

By carrying out the procedure as in stage 2 of Example 5, but using 267 mg of 5-methoxy-1-phenyl-1H-indole-2-carboxylic acid, 211 mg of 1-(3-dimethylaminopropyl)-3-ethylcarbodiimide hydrochloride (EDCl), 13 mg of 1-hydroxybenzotriazole hydrate (HOBT) and 244 mg of 1-(3,5-dimethoxyphenyl)piperazine in 15 ml of dichloromethane; after purification by flash chromatography on silica gel (70-230 mesh), elution being carried out with a mixture of dichloromethane and ethanol (98.5-1.5 by volume), followed by recrystallization from 15 ml of diethyl ether, 400 mg of [4-(3,5-dimethoxyphenyl)piperazin-1-yl](5-methoxy-1-phenyl-1H-indol-2-yl)methanone are obtained in the form of light beige crystals, the characteristics of which are as follows:
mass spectrum (EI): m/z=471 (M⁺)
melting point (Kofler bench)=165° C.

### EXAMPLE 10

### [4-(3-chlorophenyl)piperazin-1-yl](5-methoxy-1-phenyl-1H-indol-2-yl)methanone

By carrying out the procedure as in stage 2 of Example 5, but using 267 mg of 5-methoxy-1-phenyl-1H-indole-2-carboxylic acid, 211 mg of 1-(3-dimethylaminopropyl)-3-ethylcarbodiimide hydrochloride (EDCl), 13 mg of 1-hydroxybenzotriazole hydrate (HOBT) and 216 mg of 1-(3-chlorophenyl)piperazine in 15 ml of dichloromethane; after purification by flash chromatography on silica gel (70-230 mesh), elution being carried out with a mixture of dichloromethane and ethanol (98.5-1.5 by volume), followed by recrystallization from 15 ml of diethyl ether, 450 mg of [4-(3-chlorophenyl)piperazin-1-yl](5-methoxy-1-phenyl-1H-indol-2-yl)methanone are obtained in the form of beige crystals, the characteristics of which are as follows:
mass spectrum (EI): m/z=445 (M⁺)
melting point (Kofler bench)=125° C.

### EXAMPLE 11

### [4-(3-chlorophenyl)piperazin-1-yl](5-chloro-3-phenyl-1H-indol-2-yl)methanone

By carrying out the procedure as in stage 2 of Example 5, but using 100 mg of 5-chloro-3-phenyl-1H-indole-2-carboxylic acid, 77 mg of 1-(3-dimethylaminopropyl)-3-ethylcarbodiimide hydrochloride (EDCl), 54 mg of 1-hydroxybenzotriazole hydrate (HOBT) and 73 mg of 1-(3-chlorophenyl)-piperazine in 15 ml of dichloromethane; after purification by flash chromatography on silica gel (70-230 mesh), elution being carried out with a mixture of cyclohexane and ethyl acetate (50-50 by volume), followed by crystallization from 3 ml of diisopropyl ether, 110 mg of [4-(3-chlorophenyl)piperazin-1-yl](5-chloro-3-phenyl-1H-indol-2-yl)methanone are obtained in the form of a beige solid, the characteristics of which are as follows:
mass spectrum (EI): m/z=450 (M⁺)
melting point (Kofler bench)=188° C.

### EXAMPLE 12

### [4-(3,5-dimethoxyphenyl)piperazin-1-yl](2-phenylimidazo-[1,2-a]pyridin-3-yl)methanone

466 mg of 1-(3,5-dimethoxyphenyl)piperazine, 443 mg of 1-(3-dimethylaminopropyl)-3-ethylcarbodiimide (EDCl) and 312 mg of 1-hydroxybenzotriazole hydrate (HOBT) are added to a solution of 500 mg of 2-phenylimidazo[1,2-a]pyridine-3-carboxylic acid, which can be prepared according to J. of Heterocyclic chemistry (1989), 26(6), 1875-80, in 70 ml of dichloromethane. After stirring at ambient temperature for 20 hours, the reaction mixture is washed with water, dried over magnesium sulfate and concentrated under reduced pressure. The residue obtained is purified by flash chromatography on silica gel (60; 30-75 μM), elution being carried out with a mixture of dichloromethane and methanol (99/1 by volume); 632 mg of [4-(3,5-dimethoxyphenyl)piperazin-1-yl](2-phenylimidazo[1,2-a]pyridin-3-yl)methanone are thus obtained in the form of a white foam, the characteristic of which is as follows:
mass spectrum (EI): m/z=442 (M⁺).

### EXAMPLE 13

### 3-[4-(2-phenylimidazo[1,2-a]pyridine-3-carbonyl)piperazin-1-yl)benzamide

117 mg of 1-(3-carboxamidophenyl)piperazine dihydrochloride are added to a solution of 100 mg of 2-phenylimidazo[1,2-a]pyridine-3-carboxylic acid, which can be prepared according to J. of Heterocyclic chemistry (1989), 26(6), 1875-80, in 30 ml of dichloromethane, in the presence of 177 μl of triethylamine, 88.6 mg of 1-(3-dimethylaminopropyl)-3-ethylcarbodiimide (EDCl) and 62.4 mg of 1-hydroxybenzotriazole hydrate (HOBT). After stirring at ambient temperature for 20 hours, the reaction mixture is washed with water, dried over magnesium sulfate and concentrated under reduced pressure. The residue obtained is purified by flash chromatography on silica gel (60; 30-75 μm), elution being carried out with a mixture of dichloromethane and methanol (98/2 by volume); 180 mg of 3-[4-(2-phenylimidazo[1,2-a]pyridine-3-carbonyl)piperazin-1-yl)benzamide are thus obtained in the form of a white powder, the characteristic of which is as follows:
mass spectrum (EI): m/z=425 (M⁺).

### EXAMPLE 14

### [4-(3,5-dimethoxyphenyl)piperazin-1-yl](5-phenyl-1H-pyrrolo-[1,2-c]thiazol-6-yl)methanone

The procedure is carried out as in example 5, but using, firstly, 150 mg of 5-phenyl-1H-pyrrolo[1,2-c]thiazole-6-carboxylic acid, which can be prepared according to Heterocycles (2001), 55(10), 1843-1857, and 136 mg of 1-(3,5-dimethoxyphenyl)piperazine in 20 ml of dichloromethane, in the presence of 129 mg of 1-(3-dimethylaminopropyl)-3-ethylcarbodiimide hydrochloride (EDCl) and 91 mg of 1-hydroxybenzotriazole hydrate (HOBT), with stirring at ambient temperature for 24 hours. After purification by flash chromatography on silica gel (60; 30-75 μm), elution being carried out with a mixture of dichloromethane and methanol (99/1 by volume), 126 mg of [4-(3,5-dimethoxyphenyl)piperazin-1-yl](5-phenyl-1H-pyrrolo-[1,2-c]thiazol-6-yl)methanone are obtained in the form of a white powder, the characteristics of which are as follows:
mass spectrum (EI): m/z=449 (M⁺)
melting point (Kofler bench): 98° C.

### EXAMPLE 15

### [4-(3-cyanophenyl)piperazin-1-yl](5-phenyl-1H-pyrrolo[1,2-c]-thiazol-6-yl) methanone

The procedure is carried out as in example 5, but using, firstly, 150 mg of 5-phenyl-1H-pyrrolo[1,2-c]thiazole-6-carboxylic acid, which can be prepared according to Heterocycles (2001), 55(10), 1843-1857, and 159 mg of 1-(3-cyanophenyl)piperazine hydrochloride in 15 ml of dichloromethane, in the presence of 129 mg of 1-(3-dimethylaminopropyl)-3-ethylcarbodiimide hydrochloride (EDCl), 190 μl of triethylamine and 91 mg of 1-hydroxybenzotriazole hydrate (HOBT), with stirring at ambient temperature for 24 hours. After purification by flash chromatography on silica gel (60; 30-75 μM), elution being carried out with a mixture of dichloromethane and ethyl acetate (80/20 by volume), 185 mg of [4-(3-cyanophenyl)piperazin-1-yl](5-phenyl-1H-pyrrolo[1,2-c]thiazol-6-yl)-methanone are obtained in the form of a white powder, the characteristic of which is follows:
mass spectrum (EI): m/z=414 (M⁺).

### EXAMPLE 16

### [4-(3-carboxamidophenyl)piperazin-1-yl](5-phenyl-1H-pyrrolo-[1,2-c]thiazol-6-yl)methanone

The procedure is carried out as in example 5, but using, firstly, 150 mg of 5-phenyl-1H-pyrrolo[1,2-c]thiazole-6-carboxylic acid, which can be prepared according to Heterocycles (2001), 55(10), 1843-1857, and 170 mg of 1-(3-carboxamidophenyl)piperazine hydrochloride in 20 ml of dichloromethane, in the presence of 129 mg of 1-(3-dimethylaminopropyl)-3-ethylcarbodiimide hydrochloride (EDCl), 190 μl of triethylamine and 91 mg of 1-hydroxybenzotriazole hydrate (HOBT), with stirring at ambient temperature for 24 hours. After purification by flash chromatography on silica gel (60; 30-75 μM), elution being carried out with ethyl acetate, and then recrystallization from diisopropyl ether, 40 mg of [4-(3-carboxamidophenyl)piperazin-1-yl](5-phenyl-1H-pyrrolo[1,2-c]thiazol-6-yl)-methanone are obtained in the form of a beige powder, the characteristic of which is as follows:
mass spectrum (EI): m/z=432 (M⁺).

### EXAMPLE 17

### [4-(3-hydroxymethylphenyl)piperazin-1-yl](5-methyl-2-phenyl-2H-pyrazol-3-yl)methanone

Stage 1: 3.6 ml of a solution of 4N hydrochloric acid in dioxane are added dropwise to a solution of 850 mg of 4-(3-hydroxymethylphenyl)piperazine-1-carboxylic acid tert-butyl ester, which can be obtained according to patent WO 00/015609, in 4 ml of dioxane. After reaction for 20 hours, the precipitate formed is filtered off and then washed with 20 ml of petroleum ether. 770 mg of [3-(piperazin-1-yl)phenyl]methanol hydrochloride are thus obtained in the form of an amorphous brown solid, the characteristic of which is as follows:
mass spectrum (EI): m/z=192 (M⁺).

Stage 2: The procedure is carried out as in example 5, but using, firstly, 150 mg of 5-phenyl-1H-pyrrolo[1,2-c]thiazole-6-carboxylic acid, which can be prepared according to Heterocycles (2001), 55(10), 1843-1857, and 162 mg of [3-(piperazin-1-yl)phenyl]methanol hydrochloride in 20 ml of dichloromethane, in the presence of 129 mg of 1-(3-dimethylaminopropyl)-3-ethylcarbodiimide hydrochloride (EDCl), 190 μl of triethylamine and 91 mg of 1-hydroxybenzotriazole hydrate (HOBT), with stirring at ambient temperature for 24 hours. After purification by flash chromatography on silica gel (60; 30-75 μm), elution being carried out with a mixture of chloromethane and methanol (97.5/2.5 by volume), and then recrystallization from diisopropyl ether, 165 mg of [4-(3-hydroxymethylphenyl)piperazin-1-yl]5-phenyl-1H-pyrrolo[1,2-c]thiazol-6-yl)methanone are obtained in the form of a white powder, the characteristic of which is as follows:
mass spectrum (EI): m/z=419 (M⁺).

### EXAMPLE 18

### [4-(3,5-dimethoxyphenyl)piperazin-1-yl](5-phenyl-1H-pyrrolo-[1,2-c]thiazoldioxy-6-yl)methanone and

### EXAMPLE 19

### [4-(3,5-dimethoxyphenyl)piperazin-1-yl](5-phenyl-1H-pyrrolo[1,2-c]thiazoloxy-6-yl)methanone

Stage 1: 277 mg of 5-phenyl-1H-pyrrolo[1,2-c]thiazole-6-carboxylic acid, which can be prepared according to Heterocycles (2001), 55(10), 1843-1857, are suspended in 10 ml of methanol at 0° C., 1.13 g of oxone dissolved in 5 ml of water are then added and the mixture is stirred at ambient temperature for 20 hours. 50 ml of water are then added and the phase is extracted 3 times with 50 ml of ethyl acetate. After drying over sodium sulfate and concentration under reduced pressure, 250 mg of an equimolecular mixture of 5-phenyl-1H-pyrrolo[1,2-c]thiazoledioxy-6-carboxylic acid and 5-phenyl-1H-pyrrolo[1,2-c]thiazoloxy-6-carboxylic acid are obtained, which mixture is used as it is in the subsequent stage.

Stage 2: The procedure is carried out as in example 5, but using, firstly, 240 mg of an equimolecular mixture of 5-phenyl-1H-pyrrolo[1,2-c]thiazoledioxy-6-carboxylic acid and 5-phenyl-1H-pyrrolo[1,2-c]thiazoloxy-6-carboxylic acid, obtained in the preceding step, and 192 mg of 1-(3,5-dimethoxyphenyl)piperazine in 20 ml of dichloromethane, in the presence of 182 mg of 1-(3-dimethylaminopropyl)-3-ethylcarbodiimide hydrochloride (EDCl) and 128 mg of 1-hydroxybenzotriazole hydrate (HOBT), with stirring at ambient temperature for 24 hours. After purification by flash chromatography on silica gel (60; 30-75 μM), elution being carried out with a mixture of dichloromethane and methanol (98/2 by volume), by recovering the first eluted fraction, 111 mg of [4-(3,5-dimethoxyphenyl)piperazin-1-yl](5-phenyl-1H-pyrrolo[1,2-c]thiazoldioxy-6-yl)methanone are obtained in the form of an orange foam, the characteristic of which is as follows:
mass spectrum (EI): m/z=481 (M⁺);
   and by harvesting the second eluted fraction, 110 mg of [4-(3,5-dimethoxyphenyl)piperazin-1-yl](5-phenyl-1H-pyrrolo[1,2-c]thiazoloxy-6-yl)methanone are obtained in the form of an orangey-pink foam, the characteristic of which is as follows:
mass spectrum (EI): m/z=465 (M⁺).

### EXAMPLE 20

### [4-(3-hydroxymethylphenyl)piperazin-1-yl]2-phenylimidazo-[1,2-a]pyridine-3-yl)methanone

265 mg of 1-(3-hydroxymethylphenyl)piperazine hydrochloride, which can be prepared as in stage 1 of example 17, 211 mg of 1-(3-dimethylaminopropyl)-3-ethylcarbodiimide (EDCl), 465 μl of triethylamine and 148 mg of 1-hydroxybenzotriazole hydrate (HOBT) are added to a solution of 274 mg of 2-phenylimidazo[1,2-a]pyridine-3-carboxylic acid, which can be prepared according to J. of Heterocyclic Chemistry (1989), 26(6), 1875-80, in 25 ml of dichloromethane. After stirring at ambient temperature for 20 hours, the reaction mixture is washed with water, dried over magnesium sulfate and concentrated under reduced pressure. The residue obtained is purified by flash chromatography on silica gel (60; 30-75 μM), elution being carried out with a mixture of dichloromethane and methanol (99/1 by volume); 155 mg of [4-(3-hydroxymethylphenyl)piperazin-1-yl](2-phenylimidazo[1,2-a]pyridin-3-yl)methanone are thus obtained in the form of a white foam, the characteristic of which is as follows:
mass spectrum (EI): m/z=412 (M⁺).

### EXAMPLE 21

### [4-(3-cyanophenyl)piperazin-1-yl](2-phenylimidazo[1,2-a]-pyridin-3-yl)methanone

260 mg of 1-(3-cyanophenyl)piperazine hydrochloride, 211 mg of 1-(3-dimethanylaminopropyl)-3-ethylcarbodiimide (EDCl), 309 μl of triethylamine and 148 mg of 1-hydroxybenzotriazole hydrate (HOBT) are added to a solution of 275 mg of 2-phenylimidazo[1,2-a]pyridine-3-carboxylic acid, which can be prepared according to J. of Heterocyclic Chemistry (1989), 26(6), 1875-80, in 25 ml of dichloromethane. After stirring at ambient temperature for 20 hours, the reaction mixture is washed with water, dried over magnesium sulfate and concentrated under reduced pressure. The residue obtained is purified by flash chromatography on silica gel (60; 30-75 μM), elution being carried out with a mixture of dichloromethane and methanol (99/1 by volume); 280 mg of [4-(3-cyanophenyl)piperazin-1-yl](2-phenylimidazo[1,2-a]pyridine-3-yl)methanone are thus obtained in the form of a white foam, the characteristic of which is as follows:
mass spectrum (EI): m/z=407 (M⁺).
   Assessment of the Inhibition of Tubulin Polymerization

Tubulin is purified from pig brains according to published methods (Shelanski et al., 1973, Proc. Natl. Acad. Sci. USA, 70, 765-768, Weingarten et al., 1975, Proc. Natl. Acad. Sci. USA, 72, 1858-1862). Briefly, the brains are ground and centrifuged in an extraction buffer. The tubulin, present in the extract supernatant, is subjected to two successive cycles of polymerization at 37° C. and depolymerization at 4° C., before being separated from the MAPS (microtubule associated proteins) by chromatography on a phosphocellulose P11 column (Whatman). The tubulin thus isolated is more than 95% pure. It is stored in a buffer known as RB/2 30% glycerol, the composition of which is 50 mM MES-NaOH [2-(N-morpholino)ethanesulfonic acid], pH 6.8; 0.25 mM MgCl₂; 0.5 mM EGTA; 30% (v/v) glycerol, 0.2 mM GTP (guanosine 5′-triphosphate).

The polymerization of the tubulin into microtubules is monitored by turbidimetry as follows: the tubulin is adjusted to a concentration of 10 μM (1 mg/ml) in the RB/2 30% glycerol buffer, to which are added 1 mM GTP and 6 mM MgCl₂. The polymerization is initiated by increasing the temperature from 6° C. to 37° C. in a cuvette with 1 cm optical path length, placed in a Uvikon 931 spectrophotometer (Kontron) equipped with a thermostatically-regulated cuvette holder. The increase in turbidity of the solution is followed at 350 nm.

The products are dissolved at 10 mM in DMSO and added at variable concentrations (0.5 to 10 μM) to the tubulin solution before polymerization. The IC₅₀ value is defined as the concentration of product which inhibits the rate of polymerization by 50%. A product with an IC₅₀ value of less than or equal to 25 μm is considered to be very active.

A product in accordance with the invention may be of use in inhibiting the proliferation of tumor cells in vitro.

Assay for Determining the Inhibition of Proliferation of the Human Colon Tumor Line HCT116

The proliferation of HCT116 cells is evaluated by measuring the incorporation of [¹⁴C]thymidine in the following way. The HCT116 cells (from ATCC) are cultured in a DMEM medium (Gibco) which contains 10% foetal calf serum and antibiotics (1% penicillin, 1% streptomycin). To carry out the proliferation assay, the cells are seeded into 96-well Cytostar microplates (Amersham), at a rate of 5000 cells per well. The [¹⁴C]thymidine (0.1 μCi/well) and the products to be evaluated are then added. Variable concentrations of products up to 10 μM are used; the DMSO (solvent used to dissolve the products) should not exceed 0.5% in the medium. 48 hours after incubation at 37° C., the radioactivity incorporated into the cells is measured by counting the plates in a Tri-Lux counter (Wallac). The IC₅₀ value is defined as the concentration of product which reduces the radioactivity by 50% compared with an untreated control. A product with an IC₅₀ value of less than 10 μM is considered to be cytotoxic.

Biological Results

| | | | Inhibition of |
|---|---|---|---|
| | | Inhibition | HCT116 |
| | | of tubulin | proliferation |
| Example | | polymerization | IC₅₀ |
| No. | Structure | IC₅₀ (μM) | (μM) |
| 1 | 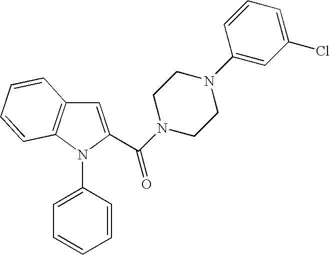 | 0.8 | 0.076 |
| 2 | 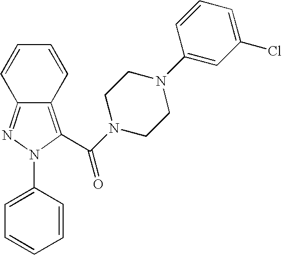 | 0.8 | 0.006 |
| 3 | 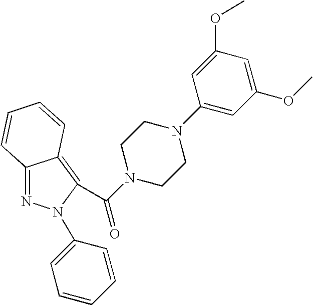 | 0.5 | 0.0098 |
| 4 | 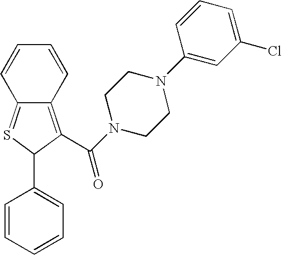 | 1.1 | 0.99 |
| 5 | 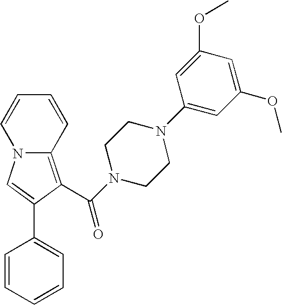 | 1.5 | 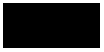 |
| 6 | 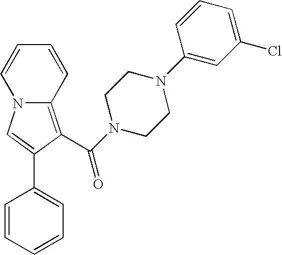 | 1.3 | 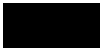 |
| 7 | 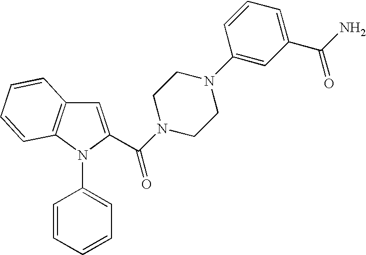 | 0.9 | 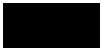 |
| 8 | 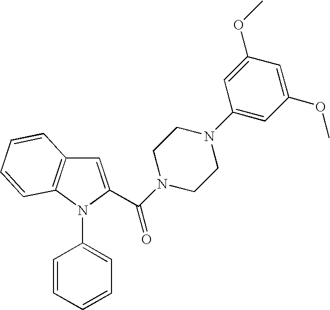 | 1.1 | 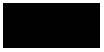 |
| 9 | 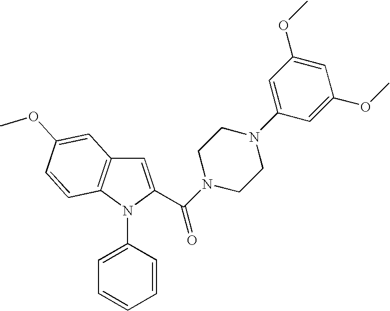 | 1.2 | 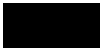 |
| 10 | 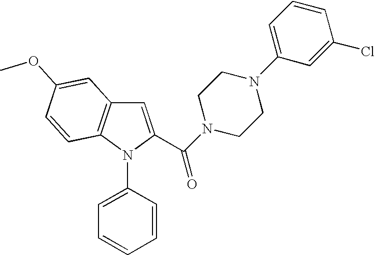 | 2.6 | 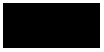 |
| 11 | 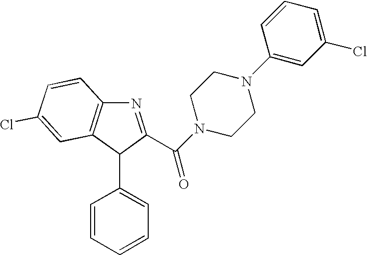 | 0.8 | 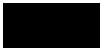 |
| 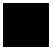 | 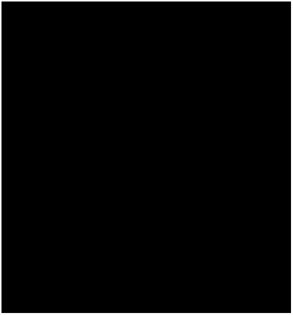 | 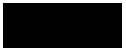 | 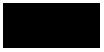 |
| 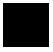 | 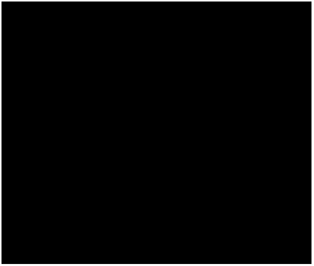 | 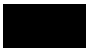 | 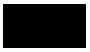 |
| 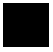 | 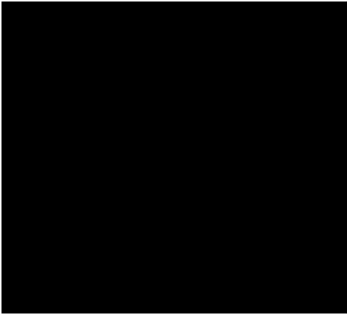 | 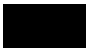 | 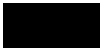 |
| 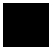 | 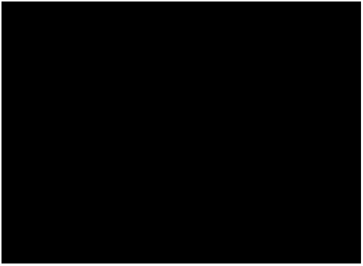 | 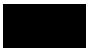 | 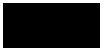 |
| 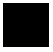 | 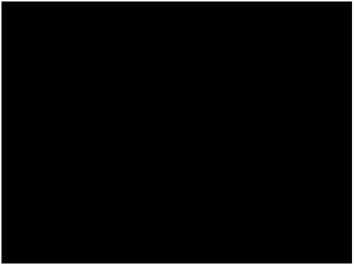 | 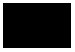 | 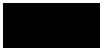 |
| 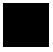 | 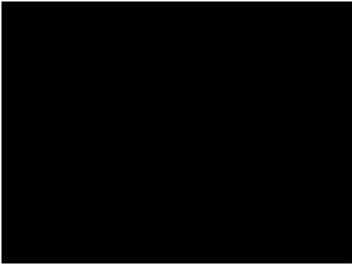 | 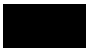 | 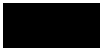 |
| 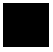 | 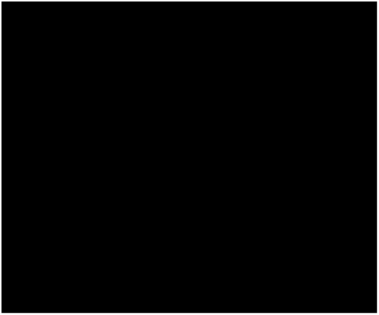 | 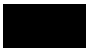 | 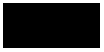 |
| 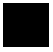 | 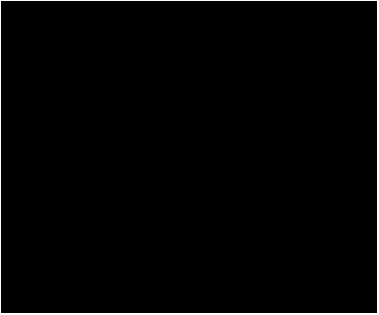 | 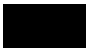 | 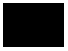 |
| 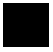 | 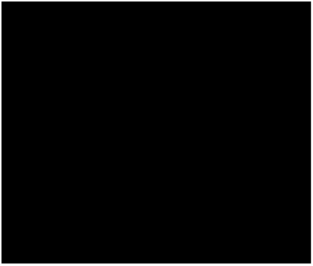 | 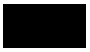 | 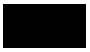 |
| 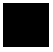 | 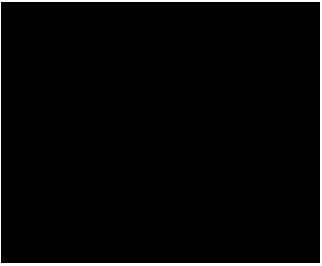 | 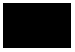 | 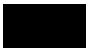 |

n.d.: not determined

1. A compound of formula (I): 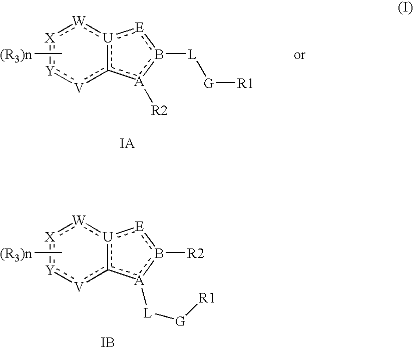
wherein:
1) (i) A, B, U, V, W, X, Y are each independently N, C or CR4; or
(ii) A, B, U are each independently N, C or CR4; V and W are CH₂, X is chosen from S, SO and SO₂; and Y is a bond;
2) L-G-R1 is chosen from 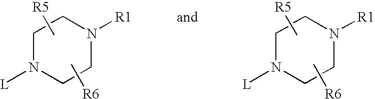
3) E is CR4, N, NR4 or S;
4) R1 and R2 are selected independently from the group consisting of aryl, heteroaryl, substituted aryl and substituted heteroaryl;
5) L is selected from the group consisting of C═O, C═S and C═N(R7);
6) R3 is selected from the group consisting of halogen, CF₃, CN, NO₂, (C₁-C₃)alkyl, (C₁-C₃)alkenyl, (C₁-C₃)alkynyl, O—R7, S—R7, SO—R7, SO₂—(R7), N(R7)(R8), halogen, CO—OR7, CO—N(R7)(R8), SO₂—N(R7)(R8), NR7-CO—R8 and NR7-SO₂—(C₁-C₃)alkyl;
7) n=0, 1, 2 or 3, it being understood that, when n is greater than 1, the radicals R3 may be identical or different, and when n=2, X and Y are not simultaneously substituted with R3;
8) R4 is selected from the group consisting of H and (C₁-C₃)alkyl;
9) R5 and R6 are selected independently from the group consisting of H and (C₁-C₃)alkyl;
10) R7 and R8 are selected independently from the group consisting of H, (C₁-C₃)alkyl and substituted (C₁-C₃)alkyl;
in the racemic form, enriched in one enantiomer, enriched in one diastereoisomer, its tautomers, its prodrugs and its pharmaceutically acceptable salts, with the proviso that the product of formula (I) is not one of the following compounds, optionally as their salts: 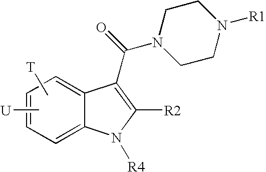
wherein:
(i) R1 is chosen from pyrid-2-yl and substituted pyrid-2-yl, each optionally in N-oxide form;
R2 is chosen from thien-2-yl, pyrid-2-yl, pyrid-3-yl, pyrid-4-yl, phenyl, phenyl substituted with at least one substituent chosen from F, OH, CF₃, Me, OMe and NO₂, wherein, when R2 is pyrid-2-yl, pyrid-3-yl or pyrid-4-yl, each optionally in N-oxide form;
R4 is chosen from methyl, 2-fluoroethyl and ethyl;
T and U are chosen independently from H, methyl, Cl and F; or else
(ii) R1 is chosen from pyrid-3-yl and pyrid-4-yl,
R2 is chosen from thien-2-yl and phenyl;
R4 is chosen from methyl and 2-fluoroethyl;
T and U are chosen independently from H, methyl, Cl and F.
(iii) R1 is pyrid-2-yl substituted in the 5-position with a tetrazolyl or amide substituent, which is optionally substituted;
R2 is phenyl;
R4 is methyl; T is 5-methyl; U is H;
(iv) R1 is pyrazin-2-yl substituted in the 5-position with CH₂CONH₂ or amide, which optionally substituted;
R2 is phenyl;
R4 is methyl; T is chosen from 5-methyl, 5-chloro, 5-fluoro and 5-bromo; U is H; 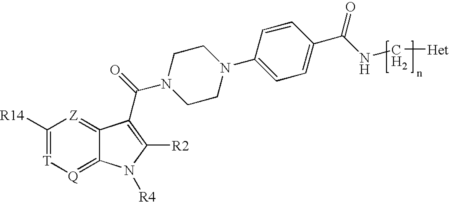
wherein:
n is 2 or 3;
Het is 4-methylthiazol-5-yl or imidazol-1-yl;
R2 is phenyl;
R4 is methyl;
T, Q and Z are chosen independently from N and CH, and R14 is H or methyl; wherein:
when T is N, then Q and Z are CH and R14 is H;
when Q is N, and T and Z are CH, then R14 is H or methyl;
and
when T is CH, then R14 is H.

2. The compound as set forth in claim 1, wherein L-G-R1 is 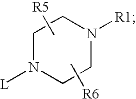
and wherein R1, R5 and R6 are as defined in claim 1.

3. The compound as set forth in claim 1, wherein A=N, B=C and E=CR4, wherein R4=H.

4. The compound as set forth in claim 1, wherein A=C, B=N and E=NR4, wherein R4=H.

5. The compound as set forth in claim 1, wherein U=N; A and B=C; E=CH; V and W are CH₂; X is SO₂; and Y is a bond.

6. The compound as set forth in claim 1, wherein R1 is chosen from:
(i) phenyl, phenyl substituted with at least one radical chosen from halogen, CF₃, CN, NO₂, (C₁-C₃)alkyl, O—R10, S—R10, N(R10)(R11), CO—O—R10, CO—N(R10)(R11) and NH—CO—R10 wherein R10 and R11 are chosen independently from H, (C₁-C₃)alkyl, halogenated (C₁-C₃)alkyl, (C₁-C₃)alkyl-OH, (C₁-C₃)alkyl-NH₂, (C₁-C₃)alkyl-COOH, (C₁-C₃)alkyl-OCH₃, (C₁-C₃)alkyl-NHCH₃, and
(ii) pyridyl and pyridyl substituted with at least one radical chosen from halogen, (C₁-C₃)alkyl, O—R12, S—R12 and N(R12)(R13), wherein R12 and R13 are chosen independently from H and (C₁-C₃)alkyl.

7. The compound as set forth in claim 6, wherein R1 is chosen from:
(i) phenyl substituted in the 3-position with a substituent chosen from halogen, (C₁-C₃)alkyl, (C₁-C₃)alkoxy, (C₁-C₃)amino, CONH₂, CO—NH—(CH₂)₂—OH, NH—CO—CH₃, and
(ii) 3-pyridyl, or
(iii) 2- or 3-pyridyl substituted with halogen, (C₁-C₃)alkyl or (C₁-C₃)alkoxy.

8. The compound as set forth in claim 1, wherein R1 is chosen from 2,3-disubstituted phenyl, 2,5-disubstituted phenyl, 3-substituted phenyl, 3,5-disubstituted phenyl, 3,4-disubstituted phenyl, 3-substituted phenyl, 3,5-disubstituted phenyl and 3,4-disubstituted phenyl.

9. The compound as set forth in claim 1, wherein R1 is chosen from 3-substituted phenyl, 3,5-disubstituted phenyl and 3,4-disubstituted phenyl.

10. The compound as set forth in claim 1, wherein R1 is chosen from 2-pyridyl, 4-substituted 2-pyridyl, 6-substituted 2-pyridyl and 4,6-disubstituted 2-pyridyl.

11. The compound as set forth in claim 1, wherein R1 is chosen from 3-pyridyl, 2-substituted 3-pyridyl and 5-substituted 3-pyridyl.

12. The compound as set forth in claim 1, wherein R1 is phenyl substituted in the 3-position with a chloro radical or a cyano radical or a carboxamido radical or a hydroxymethyl radical, or in the 3- and 5-positions with two methoxy radicals.

13. The compound as set forth in claim 1, wherein R1 is phenyl substituted in the 3-position with a CONH₂ radical.

14. The compound as set forth in claim 1, wherein R2 is chosen from 3-pyridyl, phenyl, and phenyl substituted with at least one radical chosen from halogen, alkyl, O—R10, S—R10 and N(R10)(R11), wherein R10 and R11 are chosen independently from H, alkyl and halogenated alkyl.

15. A pharmaceutical composition comprising one or more compounds of formula (I) as set forth in claim 1, in combination with a pharmaceutically acceptable excipient.

16. A method of inhibiting tubulin polymerization in a patient comprising administering to said patient a therapeutically effective amount of a compound of formula (I): 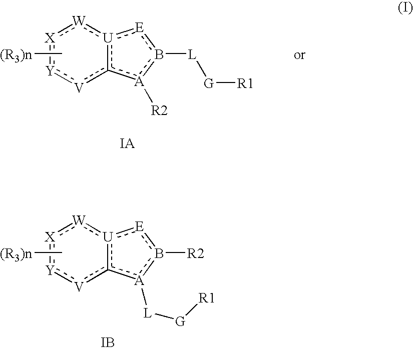
wherein:
1) (i) A, B, U, V, W, X, Y are each independently N, C or CR4; or;
(ii) A, B, U are each independently N, C or CR4; V and W are CH₂, X is chosen from S, SO and SO₂; and Y is a bond;
2) L-G-R1 is chosen from 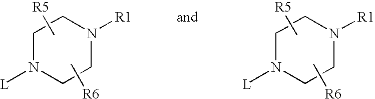
3) E is CR4, N, NR4 or S;
4) R1 and R2 are selected independently from the group consisting of aryl, heteroaryl, substituted aryl and substituted heteroaryl;
5) L is selected from the group consisting of C═O, C═S and C═N(R7);
6) R3 is selected from the group consisting of halogen, CF₃, CN, NO₂, (C₁-C₃)alkyl, (C₁-C₃)alkenyl, (C₁-C₃)alkynyl, O—R7, S—R7, SO—R7, SO₂—(R7), N(R7)(R8), halogen, CO—OR7, CO—N(R7)(R8), SO₂—N(R7)(R8), NR7-CO—R8 and NR7-SO₂—(C₁-C₃)alkyl;
7) n=0, 1, 2 or 3, it being understood that, when n is greater than 1, the radicals R3 may be identical or different, and when n=2, X and Y are not simultaneously substituted with R3;
8) R4 is selected from the group consisting of H and (C₁-C₃)alkyl;
9) R5 and R6 are selected independently from the group consisting of H and (C₁-C₃)alkyl;
10) R7 and R8 are selected independently from the group consisting of H, (C₁-C₃)alkyl and substituted (C₁-C₃)alkyl;
in the racemic form, enriched in one enantiomer, enriched in one diastereoisomer, its tautomers, its prodrugs and its pharmaceutically acceptable salts.

17. The method as set forth in claim 16, wherein said compound inhibits the proliferation of tumor cells.

18. The method as set forth in claim 16 further comprising promoting the breakup of clusters of cells originating from a vascular tissue.

19. The method as set forth in claim 16, further comprising treating cancer.

20. The method as set forth in claim 19, wherein said cancer is colon cancer.

21. The method as set forth in claim 19, wherein said cancer is rectal cancer.

22. The method as set forth in claim 19, wherein said cancer is colorectal cancer.
